(19) United States
(12) Patent Application Publication
(10) Pub. No.: US 2022/0087546 A1
(43) Pub. Date: Mar. 24, 2022

(54) VITAL SIGN CAPTURE DEVICE

(71) Applicants: Antonio Simon, Berlin, GA (US); Rodderick Spence, Doerun, GA (US)

(72) Inventors: Antonio Simon, Berlin, GA (US); Rodderick Spence, Doerun, GA (US)

(21) Appl. No.: 17/479,340

(22) Filed: Sep. 20, 2021

Related U.S. Application Data
(60) Provisional application No. 63/082,038, filed on Sep. 23, 2020.

(51) Int. Cl. A61B 5/0205; A61B 5/1172; A61B 5/1171; A61B 5/00

(52) U.S. Cl. CPC A61B 5/02055; A61B 5/1172; A61B 5/1176; A61B 5/0004; A61B 5/742; A61B 5/746; A61B 5/7405

(57) ABSTRACT

This present invention relates to a vital sign scanning apparatus that comprises a portal for individuals to pass through. The vital sign scanning apparatus detects the vital signs and other physiological conditions of a user passing through the portal which is comprised of two vertical sidewalls connected via a top wall or member. The sidewalls and/or top member are comprised of a plurality of integrated sensors including, without limitation, a heart rate sensor, SpO2 sensor, facial recognition scanner, and an infrared temperature sensor to detect the body temperature of the user passing through the portal. The sensors are in communication with a processor and a display device that displays the obtained vital sign values along with any abnormality notifications.

### CROSS-REFERENCE TO RELATED APPLICATION

The present application claims priority to, and the benefit of, U.S. Provisional Application No. 63/082,038, which was filed on Sep. 23, 2020 and is incorporated herein by reference in its entirety.

### FIELD OF THE INVENTION

The present invention relates generally to the field of health monitoring. More specifically, the present invention relates to a body scanning device used to identify health issues or other medical conditions through the monitoring or capturing of a person's pulse, blood pressure, stress level, and other physiological information and/or conditions. The body scanning device functions in a manner similar to a thermal detection unit or airport security scanner, and is capable of detecting heat from the body of an individual to identify certain conditions that may signal the presence of a sickness or illness in an individual. Additionally, the body scanning device also comprises a display that illustrates vital or other physiological signs and/or potential illness indicators for the user to view once the scan is complete. The body scanning device offers a convenient way of keeping track of an individual's vital signs and other conditions, and helps in identifying health problems and symptoms quickly and easily. Accordingly, the present disclosure makes specific reference thereto. Nonetheless, it is to be appreciated that aspects of the present invention are also equally applicable to other like applications, devices and methods of manufacture.

### BACKGROUND OF THE INVENTION

By way of background, health monitoring is very important in the early detection of illness and diseases, and can lead to a significant reduction in both suffering and medical costs. With increasing numbers of individuals having infectious diseases, and in the times of a pandemic such as COVID-19, influenza and other airborne pathogens that quickly spread from one individual to another, it is necessary to keep track of the health parameters of a population in order to monitor the health issues faced by individuals and the communities in which they live.

Individuals may easily become infected and subsequently ill due to the transmission of various germs, bacteria and other harmful pathogens present in the environment through airborne or surface contact. Due to lack of information about the symptoms, individuals may become sick, yet be unaware of what is wrong and why they are feeling ill. With a delay in symptom identification, the symptoms of a disease may reach a critical stage and the person may become chronically ill. In such scenarios, people may seek medical help and end up having to visit healthcare centers and other treatment facilities a number of times in order to properly identify symptoms. However, seeking medical advice and undergoing tests for diagnosis of a particular condition or disease may be expensive, and many individuals may not be able to afford such care. In addition, early indication of a disease may be ineffective, particularly in situations where information about a particular illness is still evolving. Additionally, people may put their health at risk if they are unable to get health issues addressed quickly by a medical professional. Further, visiting the medical health and treatment centers to seek out doctors and other healthcare professionals for diagnosis of symptoms may be both time-consuming and expensive.

Further, in public places, people may be unaware of symptoms of other people, and may easily become infected if a healthy person comes in contact with an infected person. This leads to transmission of infection and further spreading of diseases. Currently, individuals have no idea of the symptoms of other individuals in public places, such as schools, colleges, hospitals, offices, sporting and other outdoor events, large gatherings and other similar venues.

Therefore, there exists a long felt need in the art for a vital sign scan capture device that enables an individual and others to quickly and easily monitor various health parameters, and that can be used to scan and process a relatively large number of individuals, attendees or patrons at a particular event. There is also a long felt need in the art for a vital sign scan capture device that provides for the identification and tracking of various health-related parameters such as but not limited to, heart rate, blood pressure, body temperature and other physiological conditions that can be used to identify illnesses, and further prevent the spread or transmission of disease. Additionally, there is a long felt need in the art for a vital sign scan capture device that identifies symptoms quickly, thereby enabling the user to seek medical attention and prevent the detected condition from worsening. Moreover, there is a long felt need in the art for a vital sign scan capture device that eliminates the need for an individual to visit a healthcare or other treatment center for a mild symptom or condition, and that provides quick results thereby not requiring the user to wait for days or longer to know the results of the scan. Finally, there is a long felt need in the art for a vital sign scan capture device that is relatively inexpensive to manufacture and that is both safe and easy to use.

The subject matter disclosed and claimed herein, in one embodiment thereof, comprises a full-body vital sign scanner configured to detect vital signs and other physiological conditions of a user standing in or passing through the scanner. The system comprises a metal or rigid frame element, having two vertical side walls connected to a top wall or cover plate, under which the user stands or passes through for vital sign detection. A heart rate sensor is used to detect heart variability and the SpO2 of the user. A face detector is used to detect the facial features of the user, and an infrared temperature sensor scanner is provided to detect the body temperature of the user. A motion sensor is used to detect motion around the scanner. The sensors are wired together or wirelessly connected with a processor, and detect vital signs and other physiological characteristics of the user such as fingerprint, face, ear, iris, retina, palmprint, veins and others. A display device is provided to display the detected vital sign values or other physiological conditions along with abnormality notification when the sensed or detected sign or condition is outside of a predetermined range or threshold. An alarm and a LED indicator is used to indicate the operation of the scanner, and a Wi-Fi module creates a wireless channel to wirelessly transmit the vital sign or other physiological conditions, and information to a remote device or server, such as a healthcare center, treatment facility or other location with medical or healthcare personnel.

In this manner, the novel vital sign scanning device of the present invention accomplishes all of the forgoing objectives, and provides a relatively easy, convenient and quick solution to monitor and track health parameters such as pulse, blood pressure, stress level, combinations thereof and other physiological conditions, and helps to quickly identify potential health issues of those using the health portal. The vital sign scanning device of the present invention is also user friendly, as it eliminates the need to pay multiple visits to healthcare centers to seek medical advice, and reduces and, in some cases, prevents the individual from incurring expenses associated with disease diagnosis.

### SUMMARY OF THE INVENTION

The following presents a simplified summary in order to provide a basic understanding of some aspects of the disclosed innovation. This summary is not an extensive overview, and it is not intended to identify key or critical elements, or to delineate the scope thereof. Its sole purpose is to present some general concepts in a simplified form as a prelude to the more detailed description that is presented later.

The subject matter disclosed and claimed herein, in one embodiment thereof, comprises a full-body vital sign scanner configured to detect vital signs or other physiological conditions of a user standing in or passing through the scanner. The scanner includes a metal or rigid, generally upright frame, forming a portal, and has two vertical side walls connected through a top wall or cover, under which the user passes through or stands for a pre-determined increment of time in order to allow for detection of one or more vital signs or physiological conditions, such as fingerprint, face, ear, iris, retina, palmprint, veins and others. A heart rate sensor is used to detect heart variability and the SpO2 of the user. A facial recognition device is used to detect the facial features of the user. An infrared temperature sensor is provided to detect body temperature of the user, as well as other potential hot spots a user may have, thereby suggesting an area of inflation. A motion sensor to detect movement around the scanner in order to activate the scanner and begin the collection of data and other information. The scanner or portal includes a plurality of sensors which are wired or wirelessly connected together and linked with a processor, to detect vital signs or other physiological conditions of the user. A display device is provided to display the collected vital sign values or physiological conditions along with an abnormality notification when a sign or condition is collected that falls outside of a predetermined range or ranges. An alarm and an LED indicator indicate the operation of the scanner, and a Wi-Fi module creates a wireless channel to wirelessly transmit the vital sign information to a remote device or server, which may be located at a healthcare or treatment facility, or at a security office, which could then alert that action be taken to prevent a particular individual from entering or leaving an area.

In a further embodiment of the present invention, a portable vital sign scanner device is disclosed, and comprises a U-shaped frame equipped with several integrated sensors. The sensors detect a number of different conditions such as heart rate, blood pressure, and body heat to identify illnesses and combinations thereof. A digital screen is positioned on the top of the frame to display the scanned vital sign values or other collected information. The sensors are automatically activated to detect a preselected vital sign or other condition when a user stands in or passes through the U-shaped frame.

In yet a further embodiment of the present invention, a method for detecting vital signs of a user standing in or passing through a vital sign scanner device in a contactless way and notifying the user or a third party of the results is disclosed. The method comprises the steps of initially measuring heart rate variability and the SpO2 of the user using a heart rate sensor. Then, measuring the body temperature of the user using an infrared temperature sensor or sensors which may be positioned around the inner surface area of the portal to scan for body temperatures at different levels. Next, detecting details of the user using a face-detection camera to recognize various facial features of an individual that may be stored in a central database. Motion is detected around the scanner device to automatically activate the sensors and other systems in the healthcare portal. The measured vital sign values are displayed on a screen attached to the top or other convenient area of the scanner device, so that the information may be read or viewed by the individual using the portal or an agent monitoring the access point of a facility where the portal is being used. The measured vital sign values may then be transmitted to third parties such as, but not limited to, medical practitioners, healthcare workers, insurers, government officials, etc.

The advantage of the vital sign scanner device is that it identifies potential health issues through pulse, blood pressure, stress level, physiological conditions and combinations thereof. The device functions like an airport security scanner which is capable of picking up heat from the body to detect sickness or illness, and offers a way for people to keep track of body vital signs and other physiological conditions and helps to identify potential health problems that the individual may be experiencing. The vital signs and other physiological conditions such as fingerprint, face, ear, iris, retina, palmprint, veins and others, are detected in a contactless manner, without requiring a specific proximity to a person so that the measured values can be easily viewed, collected and transmitted, and further medical assistance can be sought where conditions indicate that help is required. The device can be located at any entrance or at any convenient place such as, without limitation, a mall, school, theater, business, entertainment venue or the like.

In one embodiment, the dimensions of the scanner device are 48″ W×85″ H×25″ D. In another embodiment, the dimensions of the scanner device are 50″ W×88″ H×26″ D. In one version of the scanner device, a sprinkler is present to spray or mist a sanitizer solution on the user standing in or passing through the scanner device or healthcare portal for sanitization. A sanitization reservoir may be connected through a conduit to the sprinkler or mister, and the sprinkler or mister automatically sprays or mists the sanitizing solution on the user as well as the interior area of the healthcare portal or scanner. The device has a high scanning rate and is capable of scanning or processing up to forty users per hour, with limited or no downtime.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a full-body vital sign scanner and capture device of the present invention in accordance with the disclosed architecture;

FIG. 2 illustrates a perspective view of one potential embodiment of a full-body vital sign scanner and capture device of the present invention in accordance with the disclosed architecture being used to scan an individual;

FIG. 3 illustrates a perspective and schematic view of one potential embodiment of a full-body vital sign scanner and capture device of the present invention in accordance with the disclosed architecture, wherein the device is in communication with a network and a plurality of databases;

FIG. 4 illustrates a perspective and schematic view of one potential embodiment of a full-body vital sign scanner and capture device of the present invention being used to scan an individual in accordance with the disclosed architecture, wherein the device is in communication with a network and the individual's physician, smartphone and others;

FIG. 5 illustrates a perspective view of one potential embodiment of a full-body vital sign scanner and capture device of the present invention in accordance with the disclosed architecture being used to scan an individual and being monitored by a security officer; and

FIG. 6 depicts a block diagram showing one potential method of use of the full-body vital sign scanner and capture device of the present invention in accordance with the disclosed architecture.

### DETAILED DESCRIPTION OF THE INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a vital sign scanning device that provides for the identification and tracking of various health related parameters such as, but not limited to, heart rate, blood pressure, body temperature and other physiological conditions that can be used to identify illnesses and prevent the further spread or transmission of disease. There is a long felt need in the art for a vital sign scanning device that enables an individual and others to quickly and easily monitor various health parameters, and that can be used to scan and process a relatively large number of individuals, attendees or patrons at a particular event. Additionally, there is a long felt need in the art for a vital sign scanning device that identifies symptoms quickly, thereby enabling the user to seek medical attention and prevent the detected condition from worsening. Moreover, there is a long felt need in the art for a vital sign scanning device that eliminates the need for an individual to visit a healthcare or other treatment center for a mild symptom or condition, and that provides quick results, thereby not requiring the user to wait for days or longer to know the results of the scan. Finally, there is a long felt need in the art for a vital sign scanning device that is relatively inexpensive to manufacture, and that is both safe and easy to use

The present invention, in one exemplary embodiment, is a novel full-body vital sign scanner that is configured to detect vital signs or other physiological conditions of a user standing in or passing through the scanner. The scanner or health care portal comprises a generally rigid and upright metal frame, having two vertical side walls or pedestals which are connected to one another through a top wall or cover, under which the user stands or passes through for vital sign detection and collection. The portal includes a heart rate sensor to detect heart variability and the SpO2 (pulse oximetry) of the user. A facial recognition system is used to detect facial features of the user and to compare those features to a database containing information on a number of people. One or more infrared temperature sensors are used to detect the body temperature of the user, and motion sensors are provided to detect movement around the scanner. The portal or scanner includes a plurality of sensors which may be wired or wireless connected together with a processor, which collects and compares information with a database of parameters. The sensors are used to detect vital signs and other physiological conditions of the user, and a display device is provided to display the detected vital sign values along with an abnormality notification when the collected vital signs fall outside a pre-establish normal range for that particular vital sign. An alarm and an LED light indicate the operation of the scanner and a Wi-Fi module creates a wireless channel to wirelessly transmit the vital sign and other physiological condition information to a remote device or server.

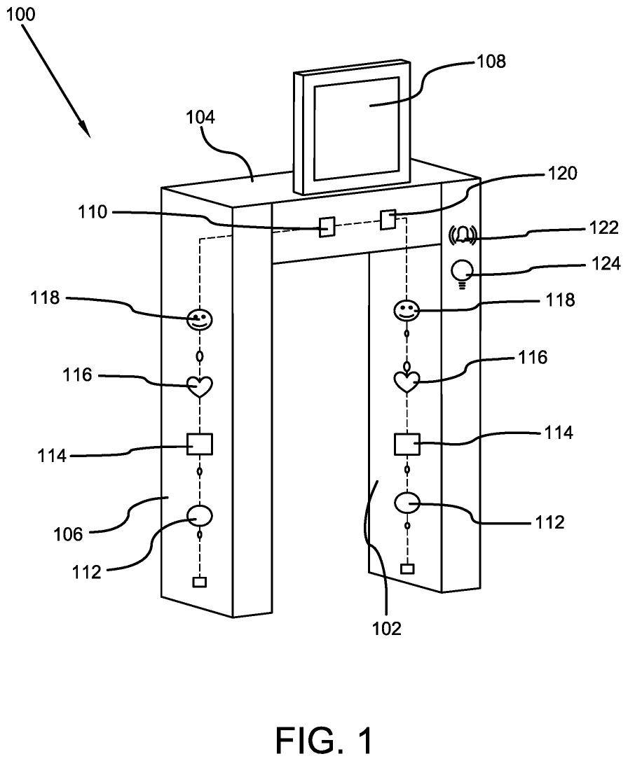

Referring initially to the drawings, FIG. 1 illustrates a perspective view of one potential embodiment of a full-body vital sign scanner and capture device 100 of the present invention in accordance with the disclosed architecture. The full-body vital sign scanner 100 is constructed like an airport security system detector having two vertical, upright walls 102, 106 connected to one another via a top or covering surface 104 that forms a covered structure. The scanner or portal provides a walk-through scanner device through which a user can easily pass through and have his or her vital signs or other physiological conditions detected by the sensors, which are embedded in the walls or top cover. The sensors are present on the interior surface of one or both of the walls and a top cover to detect vital signs or physiological conditions of a user standing between the walls 102, 106. A display 108 is attached to one surface 104 of the portal or scanner, which displays the values of the detected and collected vital signs for easy viewing by a security guard, healthcare personnel or other individual assigned to monitor the portal for possible violations or individuals of concern.

The display device 108 can be any LED or LCD touch screen showing values in a digital format along with notifications indicating an abnormality in the detected values, such as values which are outside of a particular range. The ranges can be established based on gender, age and other conditions, so that multiple parameters can be used to accurately monitor the population of individuals traversing the portal or scanner. It should be appreciated that the display device 108 can be fixed at any convenient place on the scanner device 100.

The scanner device 100 has a power source 110 to provide power to the sensors and display device 108. The power source 110 is connected to an AC power source and supports both 110 and 240V. Alternatively, the power source may be a rechargeable battery which would allow the device to be used in remote areas where connection to a traditional power source is not possible. A Wi-Fi module 120 is also present to transmit the collected vital information to a remote device, a central database or the like through a wireless communication channel.

On the interior surface of one or both the walls 102, 106, a facial recognition system sensor 118 is used to detect the identity of a user and sends the collected biometric information to a central database for authentication and storage. This is a useful sensor for businesses, schools, sporting and entertainment events, etc. where the identity of a user can be detected and compared with historical records retrieved from a central database to verify the identity of a person, thereby allowing the person access to the event or facility. A heart rate variability (HRV) sensor 116 detects the heartbeat via a microwave sensor and does not need electrodes touching the human body. Any other non-contact HRV sensor may also be used to achieve the same objective. Alternatively, a pulse oximeter may be installed to check the heart rate and oxygen saturation (SpO2) of the user.

Wireless temperature and infrared (IR) sensors 114 are used for detecting body temperature with medical precision of ±0.2° C. The wireless temperature IR sensors 114 detect skin temperature of the user standing or passing between the walls. Motion sensors 112, such as an ultrasonic motion sensor or vibration motion sensor, are present to detect motion or movement of a user near the scanner or portal to activate the unit. A processor is centrally-connected to all the sensors and sends instruction signals to the sensors. Based on the motion detection of a user, the sensors automatically activated and start detecting, scanning and collecting vital signs of the user.

An alarm speaker 122 emits a sound on completion of the vital sign collection and detection, telling the individual they may pass out of the scanner, and an LED indicator 124 indicates the status of vital sign detection and present information for the consumption of the individual. For example, a green color illumination indicates an ongoing detection and a red color illumination indicates a complete detection of vital sign.

It should be appreciated that the positioning of the sensors can be at any convenient and desired place on the interior surface of the side walls 102, 106 and top wall 104, while maintaining the efficiency of the detection of the vital signs and other physiological conditions. The display 108 gives a clear message and indication to the security staff or other medical personnel about the vital signs and physiological conditions, and any abnormality that may show up in the values which have been collected. The scanner device 100 is particularly useful for entrance points at schools, businesses, theaters, airports, entertainment venues, etc. to detect vital signs at a rapid speed in a contactless manner.

The results are synchronized with and provided to a central database, and are also shared with the user, medical practitioners to seek necessary medical assistance and security personnel. Additionally, height, weight, age and gender of the user can also be detected and video surveillance may be present. The scanner device 100 allows continuous detection of vital signs and physiological conditions of users without any downtime or significant manual effort. The scanner device 100 disclosed by the invention is simple and convenient to use, has long-term continuous work endurance, intelligent display, an abnormal physiological status alarm, wireless data transmission and the like.

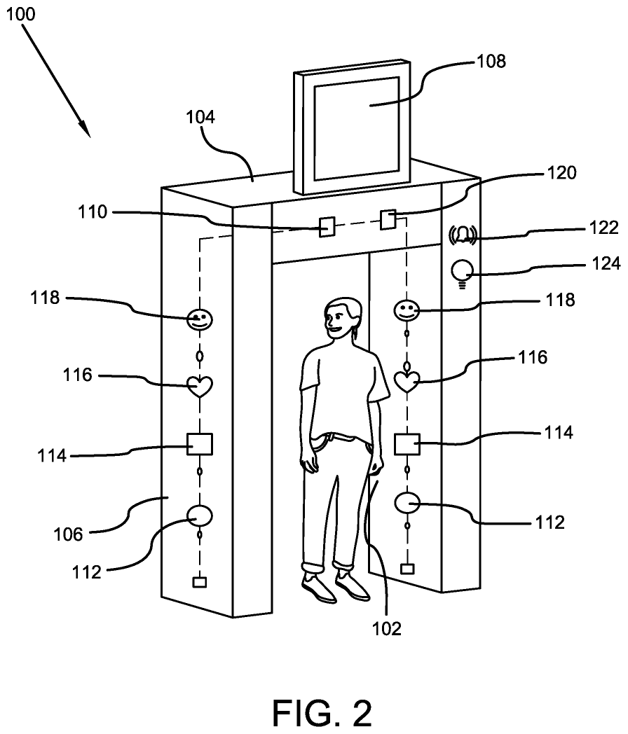

FIG. 2 illustrates a perspective view of one potential embodiment of a full-body vital sign scanner and capture device 100 of the present invention in accordance with the disclosed architecture being used to scan a user 200. More specifically, as the user 200 comes towards the scanner device 100, the motion sensor 112 detects the motion or movement and the vital signs detection sensors 114, 116, 118 and display 108 are activated. Instructions may be presented on the display indicating where the individual may need to stand in order to be fully scanned. The user 200 stands for few seconds between the side walls 102, 106 and under the top wall 104, and the activated sensors 118, 116, 114 detect the vital signs or physiological conditions of the user. The display 108 shows the detected values and other information. The alarm speaker 122 and the LED indicator 124 also indicate the completion of the process. If the displayed values on the screen 108 are normal, then the user 200 is allowed to enter the premises. Otherwise, the user may be given medical attention if required, or may be asked to leave the area. This prevents people that may have been detected as having an illness or other concerning symptoms from entering into a venue.

The scanning of vital signs or physiological conditions is done at a rapid speed using the scanner device 100 of the present invention, and the data can be transmitted wirelessly to one or more remote databases and electronic devices as described in further embodiments of the present invention. The display device 108 can be replicated onto a remote device such as a smartphone or a remote security device to remotely monitor the vital signs of the user 200.

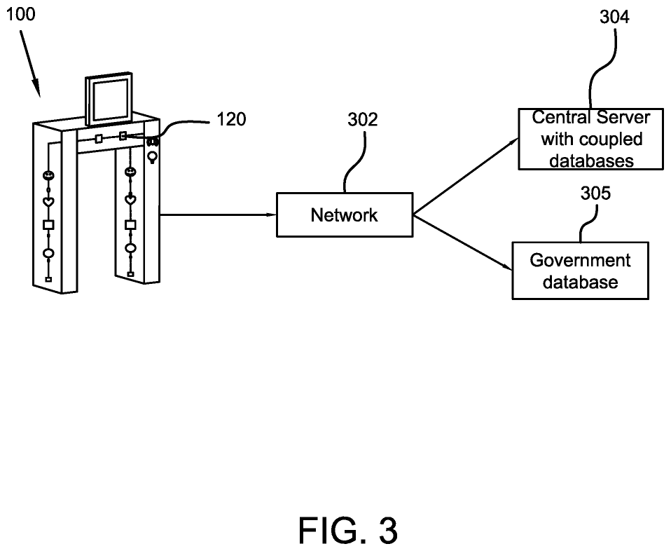

FIG. 3 illustrates a perspective and schematic view of one potential embodiment of a full-body vital sign scanner and capture device 100 of the present invention in accordance with the disclosed architecture, wherein the device 100 is in communication with a network and a plurality of databases. Through the wireless connection provided by the Wi-Fi module 120 and using the private or public network 302, the detected vital sign or physiological condition information of every user passing through the scanner device 100 is transmitted to a central server 304 of the company or database for storing the information along with the identity of the user in coupled databases. Additionally, to help the government or other sponsoring agency in tracking infectious cases of a communicable disease such as COVID-19 based on vital sign values or physiological conditions, the information is also transmitted to government databases 305 or other health services. The stored information can be tracked for making decisions about a particular user and deriving insights about the trends of infections and other relevant information.

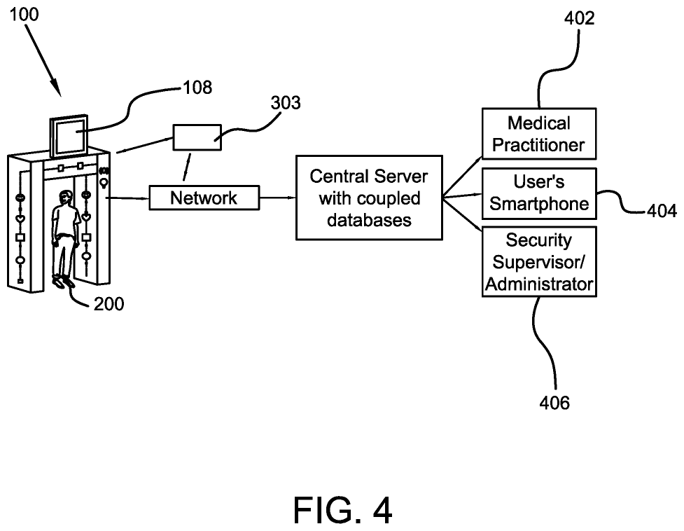

FIG. 4 illustrates a perspective and schematic view of one potential embodiment of a full-body vital sign scanner and capture device 100 of the present invention being used to scan an individual 200 in accordance with the disclosed architecture, wherein the device 100 is in communication with a network, the individual's physician, the user's smartphone and others. The detected values of vital signs by the scanner device 100 are transmitted to third parties such as a medical practitioner 402, a security supervisor or an administrator 406 for record-keeping. Based on the requirements, only the abnormal values can be transmitted to the medical practitioner 402, security supervisor or an administrator 406 for necessary action and treatment. The user also receives a notification of the detected values in the form of a Short Message Service (SMS), instant message, push notification or like. The central database 304 may store the contact information of the user along with other information such as a correspondence address, previous recordings, etc. for contacting and tracing the users.

In an alternative embodiment, a notification is provided through Bluetooth or Wi-Fi directly by the scanner device 100 to the user device. A smartphone 303 of the user may be paired with the scanner device 100 and/or the network 302, and can receive the notification. The user 200 can also view the displayed results on the display device 108. The display device 108 may have a display on both sides to allows users to view the vital sign.

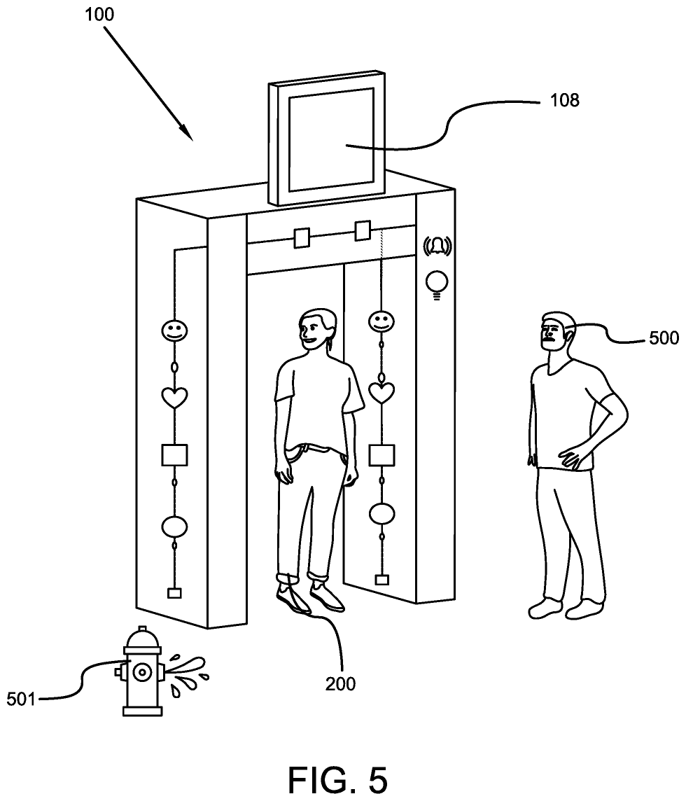

FIG. 5 illustrates a perspective view of one potential embodiment of a full-body vital sign scanner and capture device 100 of the present invention in accordance with the disclosed architecture being used to scan an individual 200, and monitored by a security officer 500. More specifically, as the user 200 stands in the scanner device 100, the vital sign values are shown on the display device 108 after the detection and collection of information is completed by the sensors, typically within only a few seconds. A security officer or other personnel 500 may then easily view the displayed values, and accordingly allow or prohibit the entry of the user 200 into the venue or facility. The display device 108 can be replicated onto any electronic device of the security officer as well for additional convenience.

The scanner device 100 provides fast, accurate and self-service sensor based vital sign collection and scanning in just a few seconds. The device 100 may provide on-board or server-based data reporting to store data. The scanner device 100 may be made up of an ABS coated panel or laminated lightweight metal. A reservoir of sanitizing solution and a sprayer or mister 501 may be provided to allow sanitizing and disinfecting of the individual and the portal 100.

All the sensors are connected to each other and to the processor through a wired mechanism within the body or frame of the scanner device 100. The sensors are integrated into the portal or scanner to avoid false alarm detection. The device 100 may optionally have a Walk/Stop indicator. The processor or microcontroller provides instructions to all the sensors to start and stop detection.

In one embodiment, the dimensions of the scanner device 100 are 48″ W×85″ H×25″ D. In another embodiment, the dimensions of the scanner device 100 are 50″ W×88″ H×26″ D. In one version of the scanner device 100, a sprinkler 501 is present to spray a sanitizer or cleaning solution on the user standing in the scanner device 100. A sanitization reservoir may be connected through a conduit to the sprinkler, and the sprinkler automatically sprays the sanitizing solution on the user. The device 100 has a high scanning rate of up to 40 users per hour, with zero downtime. Further, the device is portable, lightweight and comes in various sizes and variants.

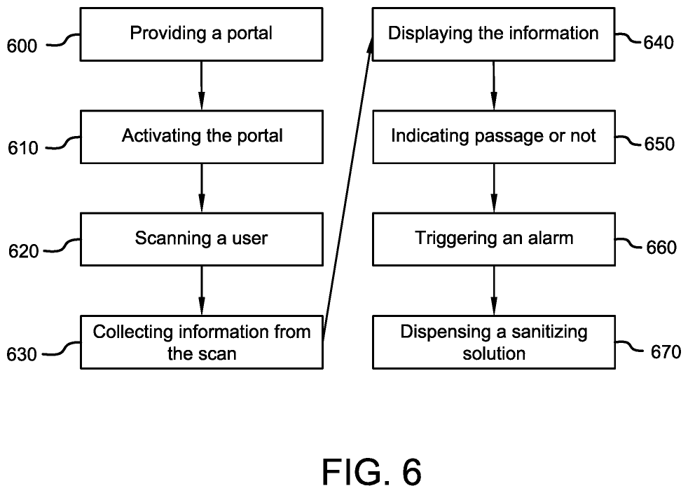

FIG. 6 depicts a block diagram showing one potential method of use of the full-body vital sign scanner and capture device 100 of the present invention in accordance with the disclosed architecture. The method begins at step 600 with the providing of the device 100. At step 610, the device 100 is activated, such as by a motion detector that senses the presence of a potential user 200 nearby. At step 620, the user 200 is scanned, and the information is collected at step 630. The collected information is then displayed on a screen at step 640, and the device 100 indicates whether or not to allow the user access to the event or venue at step 650. At step 660, an alarm, which may be audible or light-based (i.e., visual), may be used to indicated if any of the vital signs of the user 200 exceed the normal parameters from the scan. Finally, at step 670, a sanitizing solution may be dispensed to prepare the healthcare portal for the next user 200 and/or to sanitize the scanned user 200, if appropriate.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “full-body vital sign scanner”, “scanner device”, “device”, and “portable full-body vital sign scanner”, are interchangeable and refer to the full-body vital sign scanner 100 of the present invention.

Notwithstanding the forgoing, the full-body vital sign scanner 100 and its components of the present invention can be of any suitable size and configuration as is known in the art without affecting the overall concept of the invention, provided that it accomplishes the above-stated objectives. One of ordinary skill in the art will appreciate that the size, configuration and material of the full-body vital sign scanner 100 as shown in the FIGS. are for illustrative purposes only, and that many other sizes and shapes of the full-body vital sign scanner 100 are well within the scope of the present disclosure. Although the dimensions of the full-body vital sign scanner 100 are important design parameters for user convenience, the full-body vital sign scanner 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A healthcare scanning portal comprising:
a portal having a pair of generally upwardly extending walls and a connecting top or cover extending between the pair of walls to create a passageway;
a plurality of sensors integrated into at least one of the pair of walls;
a display provided on the portal; and
a processor connected to the plurality of sensors, wherein the processor is connected to at least one database.

2. The healthcare scanning portal as recited in claim 1, wherein the pair of walls comprises a first wall and a second wall that is generally parallel to, and spaced apart from, the first wall, and further wherein the plurality of sensors are integrated into both of the first and second walls.

3. The healthcare scanning portal as recited in claim 1, wherein the plurality of sensors include a heart rate sensor, a SPO2 sensor, a blood pressure sensor, a body heat sensor, a physiological condition sensor, and combinations thereof.

4. The healthcare scanning portal as recited in claim 3, wherein the physiological condition sensor senses at least one condition relating to a fingerprint, a face, an ear, an iris, a retina, a palm print, and a vein.

5. The healthcare scanning portal as recited in claim 1 further comprising a facial recognition sensor.

6. The healthcare scanning portal as recited in claim 1 further comprises a reservoir containing a sanitizing solution.

7. The healthcare scanning portal as recited in claim 1, wherein the at least one database is a governmental database.

8. The healthcare scanning portal as recited in claim 1, wherein the at least one database is one of a health care facility database or a security administrator database.

9. The healthcare scanning portal as recited in claim 1, wherein the processor collects a set of information from the plurality of sensors and the set of information is displayed on the display.

10. The healthcare scanning portal as recited in claim 9, wherein the display illustrates whether the set of information falls outside of a pre-established parameter.

11. The healthcare scanning portal as recited in claim 10, wherein an audible alarm and a LED light is activated when the set of information falls outside of the pre-established parameter.

12. The healthcare scanning portal as recited in claim 5, wherein the facial recognition sensor is in communication with at least one of a security database, a medical database or a governmental database.

13. A method of using a healthcare scanning portal comprising the steps of:
providing a portal, the portal having a first wall, a second wall and a top cover that connects the first and second walls, wherein the first and second walls comprise a plurality of sensors for scanning at least one vital sign or physiological information about an individual passing through the portal;
activating the portal via a motion sensor;
scanning the user for the at least one vital sign or physiological information;
collecting the at least one vital sign or physiological information via the plurality of sensors, wherein the plurality of sensors are connected to a processor;
displaying the at least one vital sign or physiological information received from the plurality of sensors;
indicating whether passage through the portal by the individual is allowed or not; and
triggering an alarm if the at least one vital sign or physiological information collected during the step of collecting is outside of a pre-determined range.

14. The method as recited in claim 13 comprising a further step of transmitting the at least one vital sign or physiological information to a remote database after the step of collecting.

15. The method as recited in claim 14, wherein the remote database includes at least one of a medical database, a security database or a governmental database.

16. The method as recited in claim 15 comprising a further step of conducting a facial recognition scan after the step of activating the portal.

17. The method as recited in claim 16 comprising a further step of dispensing a sanitizing solution after the step of displaying.

18. The method as recited in claim 17, wherein the step of triggering includes issuing at least one of a visual alarm or an audible alarm.

19. A scanning system for collecting a set of healthcare information, the scanning system comprising:
a portal having an opening for allowing a passage of an individual through the portal;
a plurality of sensors integrated into the portal, the plurality of sensors for collecting at least one vital sign information from the individual passing through the portal, the plurality of sensors in communication with a processor;
a motion detector for sensing when the individual is near the portal and for activating the portal to begin scanning the individual;
a display for displaying the set of healthcare information collected by the plurality of sensors; and
at least one database in communication with the portal for comparing the set of healthcare information pertaining to the individual with a set of information in the at least one database.

20. The scanning system as recited in claim 19 further comprising a facial recognition sensor.
